(19) United States
(12) Patent Application Publication
(10) Pub. No.: US 2010/0240022 A1
(43) Pub. Date: Sep. 23, 2010

(54) Reagent preparation and valving design for liquid testing

(76) Inventor: Michael R. McNeely, Murrieta, CA (US)

Correspondence Address: MORRISS OBRYANT COMPAGNI, P.C., 734 EAST 200 SOUTH, SALT LAKE CITY, UT, 84102, US

(21) Appl. No.: 12/308,486

(22) PCT Filed: Jun. 22, 2007
(86) PCT No.: PCT/US2007/014583

§ 371 Date: Dec. 16, 2008

Related U.S. Application Data
(60) Provisional application No. 60/816,056, filed on Jun. 23, 2006.

(51) Int. Cl. C12Q 1/00; C12M 1/34; F15D 1/00; F16K 11/00

(52) U.S. Cl. 435/4; 435/287.1; 137/1; 137/872

(57) ABSTRACT

The technology described in this disclosure is a combination of controlled and precise ‘reagent delivery’ integrated together with controlled liquid flow through a sample processing device used for generating a desired chemical or biological reaction.

### CROSS-REFERENCE TO RELATED PATENT APPLICATIONS

This PCT application claims benefit and priority as a Continuation-In-Part of PCT Application Serial No. PCT/US2006/005889 filed on Feb. 16, 2006, titled “LIQUID VALVING USING REACTIVE OR RESPONSIVE MATERIALS”, which in turn claims benefit and priority to the filing of U.S. Provisional Patent Application Ser. No. 60/653,566 filed on Feb. 16, 2005, titled “LIQUID VALVING USING REACTIVE OR RESPONSIVE MATERIALS” and also the filing of U.S. Provisional Patent Application Ser. No. 60/674,476 filed Apr. 25, 2005, titled “LIQUID VALVING USING REACTIVE OR RESPONSIVE MATERIALS” the contents of all of which are incorporated herein by reference for all purposes. This PCT application further claims benefit and priority to the filing of U.S. Provisional Patent Application Ser. No. 60/816,056 filed on Jun. 23, 2006, titled “REAGENT PREPARATION AND VALVING DESIGN FOR LIQUID TESTING” the contents of which are incorporated herein by reference for all purposes.

### BACKGROUND OF THE INVENTION

1. Field of the Invention

The present invention relates generally to devices and methods for performing single or multi-step biological or chemical assays on liquid samples. More specifically, the present Invention relates to valving mechanisms and reagent preparation methods used for performing these assays, and structural members for containing same and related methods.

2. Description of Related Art

Typical biological or chemical assays are performed by reacting known reagents with an unknown sample. Multi-step assays generally require the product of one reaction to come to completion and then this product is caused to react or mix with secondary or tertiary reagents to complete the assay.

This disclosure describes how many of these single or multi-step biological or chemical assays can be performed in a disposable, integrated and easy-to-use platform.

### SUMMARY OF THE INVENTION

The technology described in this disclosure is a combination of controlled and precise ‘reagent delivery’ integrated together with controlled liquid flow through a sample processing device used for generating a desired chemical or biological reaction. The ‘reagent-delivery’ is primarily achieved through reagent preparation processes such as those used in pharmaceutical manufacturing. Controlled liquid flow is primarily achieved through various valving methodologies and configurations. Multiple chemical or biological reactions on aliquots of a single sample are generally performed in parallel within one device. The results of the chemical or biological reactions are generally easily read or identifiable to the user with or without the air of a separate instrument interfaced with the device. The manual processes required to complete the reactions are generally very simple, straight forward and minimal.

### BRIEF DESCRIPTION OF THE FIGURES

The following drawings illustrate exemplary embodiments for carrying out the invention. Like reference numerals refer to like parts in different views or embodiments of the present invention in the drawings.

FIGS. 1A-B show a testing kit with four reaction chambers and an integrated sample collection cup, respectively, according to embodiments of the present invention.

FIG. 2 shows a device with a positive and negative color control chamber and chambers for six different potential titration concentrations of a particular analyte according to an embodiment of the present invention.

FIG. 3 shows a single sample—single test device with an integrated ampule for liquid reagent delivery according to an embodiment of the present invention.

FIG. 4 illustrates a single main flow path being split into two flow paths leading to two valves according to an embodiment of the present invention.

FIG. 5 illustrates a multi-step assay test kit with four parallel reaction paths and an integrated ampule encasing a liquid reagent according to embodiments of the present invention.

FIG. 6 shows the use of a plunger-like device used for delivering liquid reagent encased within a bladder to four parallel reaction paths in a multi-step assay test kit according to embodiments of the present invention.

FIG. 7 illustrates the design of a test kit with an integrated syringe fitting and plunger device for delivering a liquid sample and a liquid reagent into a multi-step assay test kit according to embodiments of the present invention.

FIG. 8 shows the use of liquid reagents stored in reaction chambers and how this can be achieved by the use of check-valves for controlling the direction of air and liquid flow according to embodiments of the present invention.

FIG. 9 illustrates how check-valves can be used to allow an integrated aspiration bulb stroke-volume to be smaller than the total volume needed to fill a device according to embodiments of the present invention.

FIG. 10 illustrates a test kit device using a check-valve to hold a secondary liquid reagent retained within a well but not encased within a bladder or ampule, for use in a multi-step assay according to embodiments of the present invention.

FIG. 11 illustrates a test kit device using on-board liquids to prime temporary valves to better improve their timing capability, and bypass channels with permanent valves to allow sample fluid to advance during the temporary valve priming process according to embodiments of the present invention.

FIG. 12 illustrates a test kit device used for multi-step ELISA-based diagnostics according to embodiments of the present invention.

FIG. 13 shows a test kit design which allows four different reaction conditions to be present in four parallel flow paths according to embodiments of the present Invention.

FIG. 14 illustrates a design useful for concentrating the analyte of interest from a dilute sample and delivering the concentrated analyte into a sample processing system according to an embodiment of the present invention.

FIG. 15 illustrates a test kit design for concentrating an analyte from a dilute sample using only a hand pump to filter the sample through a capturing region, and then draw in a release reagent stored on-board to deliver the analyte to a downstream processing system according to an embodiment of the present invention.

### DETAILED DESCRIPTION OF THE INVENTION

Embodiments of the present invention are not meant to be limited by the nature of the liquid sample or the nature of the chemical or biological reactions that are to take place, or the method of detecting the reaction products. This disclosure is not meant to be limited by the sample volumes, testing kit or channel or reaction chamber dimensions, or materials from which the testing kit is made or the materials used to perform the valving methods described.

The two primary components of the technology disclosed in the present invention are a ‘valving’ technology designed to control sample aliquoting and sample movement within the device, and a ‘reagent delivery’ technology.

The ‘valving’ technology is described in part in PCT/US2006/605889, which is incorporated herein by reference for all purposes. It consists primarily of permanent, temporary and time-delayed valves and valving systems for achieving the desired fluid control needed for a specific reaction or set of reactions to take place.

This valving technology relies on the absorption or reaction of valve materials with the sample liquid to generate a product that impedes further flow of the sample liquid.

Suitable valving materials include hydrogel powder and high-viscosity carboxymethyl cellulose salts for aqueous liquids, and polystyrene, polyamide, polymethylmethacrylate (PMMA) and acrylonitrile butadiene styrene (ABS) powder for organic liquids, petroleum products or solvents.

An additional valving methodology is described herein that encompasses the use of ‘check-valves’ to control the flow of liquid or air within a testing device, or to retain liquid reagents within specified areas in the device.

The previous disclosure referred to also describes manufacturing methods and materials for these devices, and reaction product detection methodologies.

The ‘reagent delivery’ technology is primarily achieved by making reagents ‘available’ for reactions in a controlled manner and in a controlled sequence, even though several reagents may be stored and present within the same reaction area simultaneously. The control of reagent availability is achieved through timed or stimulated reagent release, and/or reagent activation.

Reagents are generally stored in a dried format. They may be in compressed powder form, or present as lyophilized pellets, coated beads, or test strips. Dried reagents may also be coated with soluble coatings designed to delay their release, or embedded within swellable materials for the same effect. The reagents may also be liquids stored in soluble capsules, or in breakable ampules or bladders.

In all cases care must be taken that any reagent additives or valve materials do not adversely impact the desired chemical or biological reactions. In many cases valve materials are actually downstream from the reaction site and their involvement in the reaction is negligible. An exception to this is a flow-through, isolation, or time-delayed valve, which may be upstream of the reaction chamber.

Timed Reagent Release: Reagents are ‘delivered’ to a reaction area by causing dried reagents stored in the reaction area to dissolve in the incoming sample liquid. Dissolving allows the reagent to become ‘active’ and ‘available’ for reaction. Solubility of reagents in the sample liquid can be modified to be very fast or very slow, or fast but delayed for a specific time.

Adjusting the solubility rate is primarily achieved by altering the grain size of dried reagents, by altering the porosity of solid reagent tablets, or by some other means designed to increase the surface area of the reagent. The larger the surface area of the reagent exposed to the sample liquid, the faster it will dissolve in the sample liquid.

Increasing the porosity of a solid reagent tablet can be achieved by the use of rapidly dissolving filler materials, such as various sugars, various celluloses or soluble salts. When these dissolve, the porosity of the tablet increases, allowing more surface area of the slower dissolving reagent to be exposed.

Starch can be used both as a reagent binder to facilitate ‘tabletting’ of the reagents, and as a ‘disintegrant’. When the tablet is wetted by the incoming liquid sample, the starch aids in tablet disintegration, exposing active reagents stored inside to absorption.

Lyophilization, or freeze-drying, of reagents also often generates highly-porous tablets or pellets, which may be useful in this application.

Elevated temperatures also generally increase solubility. This can be achieved by mixing powder acids and bases which can cause exothermic reactions, or by dissolving certain reagents alone, which sometimes can be exothermic. Increasing the acidity or alkalinity of the sample may also increase reagent solubility. In all cases care must be taken that secondary reactions, such as to generate heat, do not adversely effect the primary reaction, and that secondary reactions do not generate excessive gases, or insoluble gases, which may interfere with device function.

In some cases multiple reactions are required to generate a product of interest. Sometimes these reactions may interfere with each other if all reagents are available simultaneously. This may be remedied by reagents being placed separately in separate reaction chambers, the product of the initial reaction being delivered to the second reaction chamber using the fluid control and valving methods described in the previous disclosure. However, in small and integrated fluidic devices It is sometimes difficult to ensure reliably and consistently that the product of one reaction in one reaction chamber is adequately transferred to a downstream reaction chamber. Sometimes the product of one reaction is diluted out by system fluid, or unreacted sample, instead of being transferred to the second reaction chamber unaffected. This may generate inconsistencies in the transfer that may affect the reliability of the results and the quality of the device.

An alternative, and potentially more reliable method, is to have all reagents needed for a final result within a single reaction chamber, but their availability for reaction is delayed or timed appropriately to generate the desired multiple-reaction product.

A method for delaying the availability of a reagent, or delaying its dissolution in the sample liquid, is to encapsulate the reagent in a soluble shell, or embed the reagent in a matrix that must first respond in some manner before it allows the reagent to be largely available. Often the availability of such reagents over time may follow a bell-shaped curve, the initial concentration usually having negligible effect on the reactions taking place.

The science and techniques associated with making reagents available for dissolution or absorption in a timed and controlled manner is well known to those of ordinary skill in the drug design and pharmaceutical manufacturing fields. Many known and widely used pharmaceutical additives, inactive ingredients or excipients, are designed for this need. Active ingredients or reagents are often compounded with these excipients to generate a desired release and absorption profile.

An example of encapsulation would be a reagent coated in a soluble shell, or encased in a soluble capsule. The delay in dissolution of the shell or capsule would delay the availability of the reagent for reaction. Suitable shell or capsule materials include various soluble celluloses, gelatin, polysaccharides, starches or sugars.

Some coatings are designed to dissolve in acidic environments and some in basic environments. This can be used to stimulate their release at different stages of a multi-stage reaction. Such coatings include Enteric Coat L100 for neutral to basic environments and Enteric Coat L30D for acidic environments, among others. Both products mentioned are manufactured by Libraw Pharma.

An example of a reagent being embedded within a matrix would be an aqueous reagent first absorbed into hydrogel granules, the swollen granules allowed to dry and shrink, then the dried granules being stored in the reaction chamber. The hydrogel must first swell in the incoming liquid before the bulk of the reagent is released or available for reaction. The rate of swelling is proportional to the grain size of the hydrogel and other factors. Care must also be taken that the absorption characteristics of the hydrogel are appropriate for the acidity, salinity, and other characteristics of the sample liquid.

An example of an application using these techniques is the titration of ethanol in saliva, as an approximate determination of blood-alcohol concentration, or BAC.

Ethanol Titration: Blood Alcohol Concentration, or BAC, is often measured as the mass, in grams, of ethanol (EtOH) present per 100 mL of liquid (either blood or water which are approximately equivalent). For example a BAC of 0.08 represents 0.08 g EtOH per 100 mL blood. The numerical value of mass of water or blood and volume of water or blood are approximately equivalent, that is 100 mL of blood approximately equals 100 g. So, BAC is also sometimes represented as a mass percent, i.e. g EtOH/100 g blood or water. So a BAC of 0.08 may be represented as 0.08%. The numerical value of BAC is not equivalent to a volume percent or particle percent.

There are several commercially available BAC measurement devices. These include ALCO-Screen™ saliva alcohol test strips by Chematics, Inc., the Q.E.D.® Saliva Alcohol Test by OraSure Technologies Inc., and the AL-5000 Alcoscan Breathalyzer by Sentech Technologies. These include devices that approximate BAC from alternative sources to blood, such as exhaled breath and saliva. There are strong correlations between BAC and these alternative substrates. Some disadvantages of these systems are that inexpensive test strips are often inaccurate as there is no sample volume control and the reagents can wash off, and it is often difficult to read gradual color variations, usually from light green to dark green, especially in low light conditions or if the user is impaired. Other, more accurate or sophisticated techniques, are typically more expensive or may require significant skill on the part of the user to accurately perform and read. Some instrument based systems may be inexpensive on a per-test basis, but the initial instrument cost may be prohibitive. Most instruments also require regular cleaning and calibration.

It would be beneficial to have a device that is inexpensive, readily available, easy to use, easy to read, accurate, and disposable. Such a device could prove beneficial to reducing the instances of driving while intoxicated.

One technique of measuring alcohol concentration is a method involving the oxidation of ethanol with potassium dichromate, followed by the titration of excess dichromate ion with potassium iodide. This technique has not been commercialized into a kit type device because of the toxicity of the dichromate ion and the challenges associated with unskilled users attempting to perform complicated titration processes. However, the technology described in this disclosure can be used to make such a device that is easy to use, inexpensive to manufacture, accurate, and safe.

A and B are two chemical equations representing two separate reactions involved in this technique:

2Cr₂O₇²⁻+3C₂H₅OH+16H⁺→4Cr³⁺+3CH₃COOH+11H₂O A

6I⁻+Cr₂O₇²⁻+14H⁺→3I₂+2Cr³⁺+7H₂O B

Reaction A is to take place first. The dichromate ion (Cr₂O₇²⁻) comes from powder potassium dichromate. This is combined together with ethanol (C₂H₅OH) in a solution (saliva) with excess acid (H⁺). The acid was derived from a powder form of sodium hydrogen sulfate (NaHSO₄).

The reaction causes the reactive and toxic dichromate ion, containing Chromium(VI), or Cr(VI), to be reduced to Chromium(III), or Cr(III), a less reactive and widely used form of chromium. Cr(III) is often used in dyes, pigments, and is even a nutrient important to human health.

In the second step, reaction B, the iodide ion (I⁻), coming from powder potassium iodide (KI), is added to the product of reaction A. If any unreacted Cr(VI) remains, it will oxidize the iodide ion to form iodine (I₂).

The presence of iodine in the product of reaction B can be determined by the use of starch as an indicator. Starch, which is normally white, turns dark purple or black in the presence of iodine.

With proper preparation and measurement of reagents, and proper control of the volume of the liquid sample used, the amount of dichromate added to reaction A can be made to exactly correspond to the amount needed to fully oxidize a specific concentration of ethanol in the sample solution. This would mean that no dichromate remains in excess to be available in reaction B. Hence, no iodine would form and the starch indicator would remain white.

In a reaction where the reagents are not properly measured, or where the concentration of ethanol is not known, the starch indicator remaining white indicates either too much (or exactly the right amount) of ethanol is present, or not enough (or exactly the right amount) dichromate is present. The starch indicator turning purple or black indicates that too little ethanol is present or too much dichromate is available.

Given that the mass of dichromate present can be specified, and the volume of the sample and target concentration (and hence—mass) of ethanol is known, accurate titrations can be made providing accurate (stepwise) measurement of the concentration of ethanol in the sample. All other reagents, e.g. acid and iodide ion, are available in excess so that they are not limiting factors in the reaction.

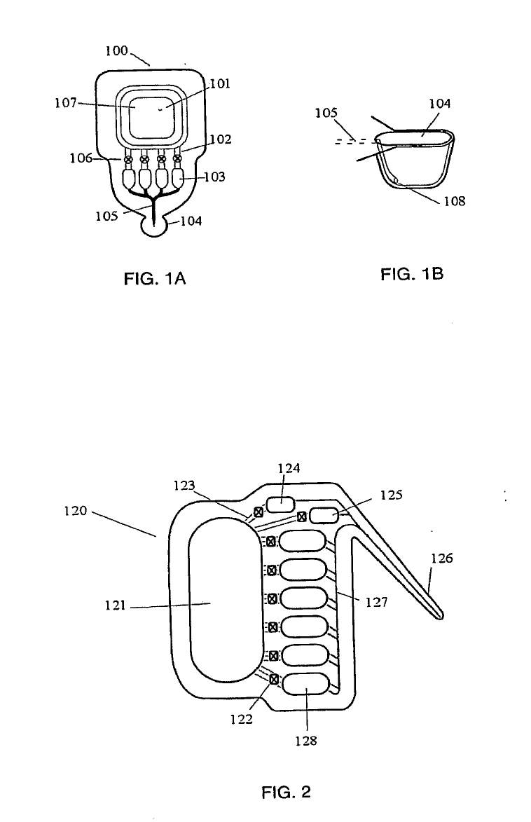

FIG. 1A illustrates a device 100 containing four reaction chambers 103 designed to react with four separate aliquots of a single saliva sample. Device 100 may include an integrated bulb 107 for sample loading, and an integrated cup 104. FIG. 1B illustrates the integrated cup for sample collection in perspective view. One of the four reaction chambers 103 may contain the exact amount of dichromate needed to react with a mass of ethanol corresponding to a concentration of 0.08% BAC. A second of the four wells 103 contains the exact amount of dichromate needed to react with a mass of ethanol corresponding to a concentration of 0.06% BAC. A third well 103 contains the amount of dichromate needed to react with the ethanol in saliva sample corresponding to a concentration of 0.04% BAC. The fourth well 103 may be a control well that is designed to test the viability of reagents regardless of the actual concentration of ethanol present. That is, it turns from white to black regardless of the concentration of ethanol in the saliva.

Device 100 in FIG. 1A is operated by the user first covering the pressure release hole 101 with one finger, while simultaneously compressing the integrated aspiration bulb 107. The user then deposits a small amount of saliva in the sample receptacle or integrated cup 104, then slowly releases pressure on the aspiration bulb 107 while continuing to cover the pressure release hole 101. The saliva is drawn into the device 100 through the inlet 108 (FIG. 1B) of the main flow channel 105 (FIGS. 1A and 1B) and into the four reaction chambers 103. As the saliva enters and fills the reaction chambers 103, it will enter the air ducts 102 and enter the permanent valves 106. Once wetted by the saliva, the permanent valves 106 will immediately seal and prevent further flow into the aspiration bulb 107. When the user has identified that all reaction chambers are filled, pressure can be completely released on the aspiration bulb 107 and the pressure release hole 101 can be uncovered. Note that the presence of a pressure release hole 101 is not necessary for all applications, and will depend on the materials from which device 100 is made of and the actual valve configuration. In many instances, the valves 106 can easily withstand any pressure that may be remaining on the aspiration bulb.

In this example, the following table shows the possible results and failure indication modes of a testing kit with this design, for various levels of BAC.

| Wells | control | 0.08 | 0.06 | 0.04 |
|---|---|---|---|---|
| Before use: | w | w | w | w |
| After use, if BAC: | | | | |
| <0.04 - | b | b | b | b |
| ≧0.04, <0.06 - | b | b | b | w |
| ≧0.06, <0.08 - | b | b | w | w |
| ≧0.08 - | b | w | w | w |

Failure Indicators

| | |
|---|---|
| Before Use: | any well dark |

| | | | | |
|---|---|---|---|---|
| After Use: | | | | |
| Failure 1- | w | x | x | x |
| Failure 2- | b | w | b | b |
| Failure 3- | b | b | w | b |

Where:
b - black or purple w - white x - any color

A positive indication of the Before Use failure mode means humidity has entered the kit while stored, or some other factor has caused the reagents to spoil. After Use Failure 1 also indicates a reagent malfunction, such as improper reagent loading, or a fluid control problem. Failures 2 and 3 suggest improper fluid flow within the device.

In normal titration procedures involving this process all reagents are in liquid form and prepared, with their quality and concentration verified. Reaction A is performed first, and then the reactants for reaction B are added to the products of reaction A.

However, because all reagents are readily soluble and can be quality tested separately and measured accurately, there should be no reason why the reagents cannot be used in their respective powder forms and added to appropriate aliquots of the liquid sample. Also, if a means were devised where the reagents for reaction B, i.e. KI, were designed to have a time delay before becoming available for reaction B, then all dried reagents for both reactions could be present in the same reaction chamber simultaneously.

The two reactions must take place separately because the oxidation of the iodide ion in reaction B is competitive with the oxidation of ethanol in reaction A. Any presence of the iodide ion in reaction A can prematurely form iodine and the measurement of excess dichromate in reaction B would be inaccurate.

The key for this device to function properly is to both accelerate reaction A, from what may normally be a slow reaction, and time-delay reaction B. Reaction A can be accelerated by increasing the surface area of dichromate available for reaction, and by having excess acid available to push the reaction to completion, among other potential means. Reaction B can be delayed by having the iodide ion bound in a matrix that delays its availability, among other potential means.

The surface area of dichromate is increased by mixing powder potassium dichromate into a paste of starch (ACS Reagent S-9765) and deionized water. The starch needs to be present anyway as an indicator for the presence of iodine. The paste is spread out and (quickly) dried forming wafers or chips of the reagents. This way, instead of having clumps of accumulated powder potassium dichromate, the potassium dichromate is spread out in thin sheets, greatly increasing its surface area. In addition, as saliva contains enzymes designed to break down starch, any dichromate ‘hidden’ within the thickness of the starch is rapidly exposed. To increase shelf life of the test kit, such as to prevent degradation of the reagents and reduction of the Cr(VI), the wafers or chips need to be protected from moisture, such as by vacuum sealing.

To prepare the iodide, potassium iodide is first dissolved in water to form a concentrated solution. Hydrogel granules (HGL) are then added to the solution. The HGL swells and absorbs the potassium iodide solution. The HGL is then removed from the solution and dried. Upon drying the HGL shrinks and again forms small solid granules. It may also form a thick hard film, which can be broken up into granules. The KI-HGL granules can also be washed in a solvent, such as anhydrous alcohol, to remove any iodide present on the granules' surface. Care must be taken that the HGL used is able to swell in highly acidic solutions as will be present in the reaction chamber. Such HGLs include Enviro-Bond™ 300C by Petroleum Environmental Technologies, Inc.

The mass or concentration of dichromate in the wafers can be changed to account for the different amounts of dichromate needed to react with different target concentrations of ethanol.

Although in large amounts dichromate, or more specifically Cr(VI), is considered a health hazard, In the amounts needed to titrate the low concentrations of ethanol in the small volumes of sample, it has no known acute measurable health effect. For example, consider a BAC of 0.08, the legal limit in many countries, and a sample volume of 50 μL. A mass of approximately 300 μg potassium dichromate would be needed to react with this target concentration of ethanol. This is approximately 10,000 lower than published acute toxicity levels for potassium dichromate. In addition, the reagents are completely enclosed in the testing device, the Cr(VI) is bound in starch and, upon exposure to the atmosphere (humidity), the Cr(VI) will begin to degrade to Cr(III) automatically. The actual likelihood of acute toxicity is extremely low, and the potential for chronic toxicity is near zero.

The benefit of this type of device is its ease of use (squeeze a bulb and dispense saliva onto a small cup, both the bulb and cup are integrated into the device, and then release bulb). Reading the device is straightforward (black or white color determinations) and no expensive materials or processes are used to manufacture the device, allowing it to be very affordable.

A few potential challenges are the prevalence of bubbles in saliva samples, which could be eliminated by proper device design, and the fact that dichromate is not necessarily specific to the oxidation of ethanol in a saliva sample. Other components of the saliva may also be oxidized, although this could be reduced by ensuring the user does not eat or drink for a short time period before using the device.

Most EtOH measurement kits are rate limited and have a timeframe in which they should be read, or a ‘measurement window’. This is also the case with this design, although the measurement window could be made to be very accommodating, such as between 2 and 10 minutes.

Reagent Modification: The reagents for some reactions may not be directly soluble in the liquid to be tested. In many reactions reagents are prepared separately, sometimes by dissolving active ingredients in solvents, which are then added to the liquid sample in a later step. Although possible through the use of breakable ampules, or by performing an external preliminary sample preparation step, or by delivery of an external secondary liquid to the device, the technology described in this disclosure is most valuable when the reagents are stored in a dried format directly within the device, making the use of the device as simple as possible.

To allow some reagents, which may not be directly soluble in the sample liquid, to dissolve in the sample liquid, they may need to be modified from their standard form to improve their solubility. This can be done prior to their storage within the device, or during the reaction within the device as it is being used. In any event the reagent must maintain its primary functionality for use in the reaction, at least for the time period important for the reaction and determination of results to be completed.

For example, some organic dyes are used as indicators in chemical reactions. If the sample to be tested is aqueous, the organic dye will often not dissolve directly in it. For this reason the dyes are often dissolved in organic solvents prior to their use. The resulting solvent is then added to the aqueous sample, in which it is soluble.

When one considers the structure of the organic dye, many of them are complex organic molecules with various reactive sites. Some of the reactive sites are used in the reaction of interest for indicating the reaction has taken place, or indicating the presence of a particular product. Some reactive sites may not be involved in generating the indication of interest. These are referred to as ‘uninvolved’ reactive sites. It may be possible to make an organic dye soluble in an aqueous solution by altering its molecular structure to make it polar or ionic. That is, it can be changed from a typically complex organic dye molecule with covalent bonding, to an organic salt. This may be done by removing or altering one of the ‘uninvolved’ reactive sites. This may also be done without adversely affecting the dye's primary chemical reactivity and indicator function, at least for the time period and conditions important to the reaction and determination of reaction results.

An example of how this is done is given in the example of rhodanine dye as used in the titration of cyanide in water.

Reverse Titration of Cyanide in Water: The fatal range of cyanide (CN⁻ or just CN) poisoning for humans is approximately 250 ppm, but varies between 30 and 300 ppm depending on the amount ingested. What would be ideal for a cyanide detection device would be an inexpensive and easy to use product that is capable of detecting cyanide by untrained and inexperienced users within the concentration range of 1 to 500 ppm.

There are three primary ways cyanide is detected in water. One is colorimetric that involves the careful preparation of the water sample and the use of a spectrophotometer to detect slight variations in the sample color. It is generally useful for samples containing less than 0.5 ppm CN. Samples above this amount cause saturation in the spectrophotometer reading and must be manually diluted to bring within instrumental range, further complicating sample handling and preparation.

A second method of detecting cyanide is the use of a cyanide sensitive electrode. Sample and instrument preparation is required for this technique, which involves the calibration of the electrode for the estimated cyanide concentration range of the sample, and sample preparation which includes adjusting the pH of the sample to liberate any bound or complexed cyanide ions.

A third, and very complex method, is the titration of cyanide which involves sample preparation (similar to the previous two methods), the addition of indicator reagents, and the stepwise addition of titration reagents. This generally requires skilled personnel to prepare the sample properly and to use proper titration techniques in order to obtain an accurate result. The value of this technique is that its range of sensitivity coincides well with the range of potentially toxic concentration exposure, that is, about 5 ppm and higher.

Typically, prior to CN titration, a water sample is filtered and ‘cleaned’. Cleaning means removal of any interferents that may be present, such as oxidizers, fatty acids, sulfides, carbonates and other compounds. Interferents may interfere with the titration chemistries, or bind to the CN making it unavailable for detection. The water sample is also treated with a basic solution, such as sodium hydroxide (NaOH) to increase its pH to something around 11 or higher. This is done to push any remaining bound cyanide, such as HCN, into free cyanide, or CN″.

We are assuming that the sample is drinking water that has been tainted with CN. Drinking water is normally clean and free of most interferents. Thus ‘cleaning’ the sample is not necessary although the pH still needs to be elevated.

Once the sample's pH has been elevated through the addition of NaOH solution, a dye is prepared and added to the sample. The dye is a complex organic molecule know as 5-(4-Dimethylaminobenzylidene) rhodanine, also called p-dimethylaminobenzylidene rhodanine, or just rhodanine dye.

After the addition of rhodanine dye the sample is titrated with the stepwise addition of silver nitrate solution (AgNo₃). The silver ion complexes with the CN according to this chemical equation:

Ag⁺+2CN⁻→Ag(CN)₂⁻

Once enough silver ion is added to bind to all the CN present in the solution, any remaining Ag⁺ will bind to the rhodanine dye, causing it to change from a yellow solution (sometimes called ‘canary yellow’) to a pink solution (sometimes called ‘salmon hue’).

At the point of color change the titration is stopped and the amount of silver ion that has been added, which is known, is correlated to the amount of CN that is present in the sample in a 2:1 ratio, as the chemical equation illustrates.

This process is known as a forward titration, because at the beginning of the process the dye is not yet complexed with the titrant (Ag⁺). However, because the titration is reversible and the silver-cyanide complex is chemically and thermodynamically favored over the silver-rhodanine complex, a reverse titration is also possible.

In a reverse titration, CN is added to a high pH silver-rhodanine complexed solution. The CN that is added displaces the rhodanine from the silver-rhodanine complex and forms a silver-cyanide complex. When enough CN is added to displace all the rhodanine, the solution changes from a salmon hue color to canary yellow. The reverse titration is the process that is used in this CN detection or measurement method.

Because the reagents used (silver nitrate and sodium hydroxide) are readily soluble in the sample (drinking water), and can be accurately measured and quality tested separately, powder reagents can be used instead of liquid reagents. The only exception to this is the rhodanine dye.

The complex organic rhodanine dye molecule is not directly soluble in water. It is normally prepared by dissolving it in a solvent solution, such as acetone, which is then added to the sample later. The acetone-rhodanine solution is soluble in water.

However, as described earlier, it is possible to remove one of the ‘uninvolved’ reaction sites of the rhodanine dye, making it ionic and soluble in water. In this case excess NaOH, or a base, is used to strip one of the ‘uninvolved’ reaction sites from the molecule, making the molecule ionic. The dye then dissolves in the sample and functions as a color indicator, as described. So, although the rhodanine is not directly soluble in the sample water, it is soluble in a basic aqueous solution, that is, the sample water that has reacted with one of the reagents already used in this process.

An embodiment of a device 120 using this process is shown in FIG. 2. It contains six reaction chambers 128 with the proper amount of reagents (not shown) needed to titrate specific target concentrations of CN that may be present in the liquid sample. This is done by altering the mass of silver nitrate in the reaction chambers 128. Device 120 also contains two wells 124, 125, for a positive 124 and negative 125 control, providing references of the canary yellow and salmon hue colors.

One of the six reaction chambers 128, or an additional well (not shown) that may be added to device 120, could be used to provide indications of any interference substances that may be present. As mentioned the sample is assumed to be drinking water. In many regions the quality of drinking water is closely regulated such that the presence of any interferents is usually so low that they do not pose a problem. However, in case where the device 120 is used to test something other than drinking water, or the drinking water is further tainted with substances intended to mask the presence of cyanide, or some unregulated substances are in great excess, interferent detection wells may be useful. For example, many existing water regulations provide guidelines for allowable concentrations of sulfates, but not of sulfides. Thus, in theory, a high concentration of sulfides may be present in regulated water. Sulfides do interfere with the CN titration process. However, a high concentration of sulfides usually causes the water to have a ‘swamp-like’ stench, which is noticeable and usually unacceptable to an end user. So, the sulfides are usually kept low anyway or, if present, they can be detected in the water aromatically, or by having a well in the device dedicated to their detection.

To operate the device 120 in FIG. 2, the user simply presses the integrated aspiration bulb 121, places the tip 126 of the device 120 into the liquid to be tested (not shown), then releases pressure on the aspiration bulb 121. The test liquid is drawn into the flow channels 127 of the device 120, and fills each reaction chamber 128 of the device 120. Once a reaction chamber 128 is filled, the test liquid is drawn into the air ducts 123 which lead to the permanent valves 122, which immediately seal and prevent further liquid flow. The reagents (not shown) present in the reaction chambers 128 dissolve in the test liquid and react with it and with each other. After a few seconds the resulting colors of the reaction chambers 128 are observed and the reaction chamber 128 that changes from salmon hue to canary yellow contains the amount of reagents needed to titrate the CN present in the liquid.

Liquid Reagent Storage and Use: The technology described in this disclosure primarily focuses on techniques for performing reactions with reagents stored within the device in a dried format. Methods are described in the previous disclosure about how the valving technology can be used to allow for multiple external liquids to be delivered to reaction chambers within the device. In order to facilitate ease of use, there are also applications where it is desirous that secondary liquids stored within the device are delivered to reaction chambers.

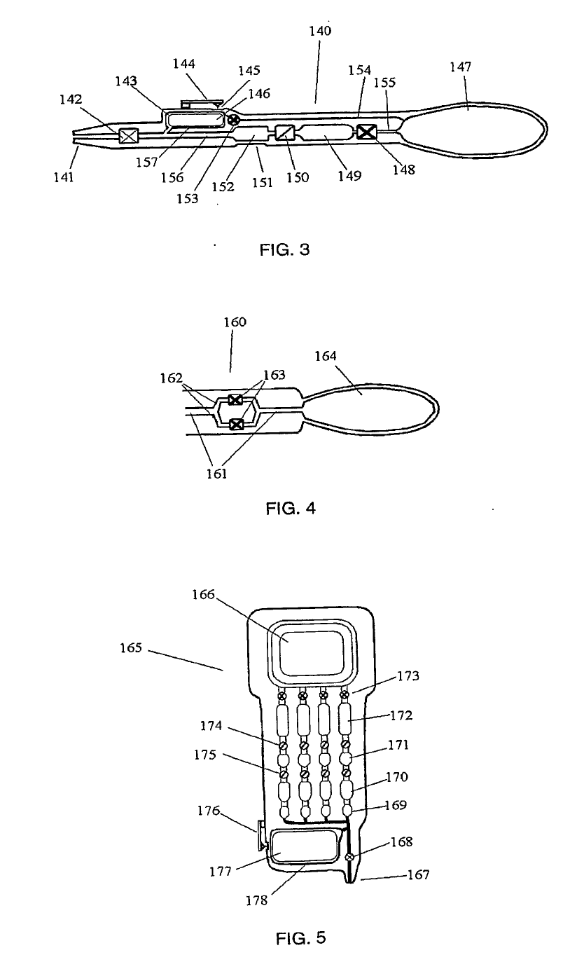

FIG. 3 shows a device 140 that illustrates a simple rendition of how an integrated, secondary liquid in well 146 can be delivered to reaction chamber 152. In this description, the primary liquid is the sample itself. The secondary liquid in well 146 may be a liquid reagent or a buffer solution or a wash solution or a reaction termination solution, or provide some other function according to various embodiments of the present invention.

In FIG. 3 the bulb 147 on one end of the device 140 is depressed and the tip 141 of the device 140 is placed in the primary solution, or sample liquid. The bulb 147 is then released, thereby drawing a vacuum at bulb channel 155. Fluid is aspirated into the device 140 where it first flows through the isolation valve 142 and into the sample reaction chamber 152. As it exits the reaction chamber 152 it encounters the temporary valve 150 which, temporarily, stops its flow. Before the temporary valve 150 releases, the isolation valve 142 completely closes. At this time the remaining aspiration force in the bulb 147 is pulling against the isolation valve 142, which is designed to withstand its force. This situation remains indefinitely until the user presses the puncture lever 144. Pressing this lever 144 both breaks a liquid filled ampule 143 stored within the device encasing the secondary liquid in well 146, and creates a secondary aspiration flow path, as the lever 144 has punctured the device wall 145 allowing air to enter the device 140. The suction force remaining in the bulb 147 draws the secondary liquid 146 into the main flow channel 156 and into the sample reaction chamber 152. Flow continues until the liquid encounters the permanent valve 148 at the exit of the dead-volume well 149. An integrated view window 151 having sufficient transparency or translucence may aid in viewing the results of the reaction in the reaction chamber 152.

Furthermore, in FIG. 3, to reduce the possibility of drawing bubbles into reaction chamber 152, the ampule 143 can be bathed in the incoming liquid sample by the use of a parallel suction path leading to the aspiration bulb through air duct 154, which joins well 157 containing the ampule 143 through the permanent valve 153. Once the Incoming liquid bathes the ampule 143 it will encounter permanent valve 153 and all remaining fluid flow will continue through the main fluid path 156.

In instances when the aspiration or dispensing force is directed onto a single flow path, such as when all remaining force is directed on flow path 156 of FIG. 3 after permanent valve 153 has sealed, or when there is only a single flow path in a device, the flow pressure or flow rates may be too great for a single valve to respond effectively. The material of the valve may be ‘blown-out’ of the valve well, or the valve may respond too slowly and a significant volume of liquid may leak out of the reaction chamber to make the reaction un-quantifiable.

The relationship between volumetric flow rate of an expanding aspiration bulb, shown as Q in the equation below, with flow cross-sectional area, A, and pressure gradient AP, is shown here:

Q∝ΔP×A EQ. 1

In a simplification of the flow dynamics of an actual system, EQ. 1 explains that the total volumetric flow rate of at system is directly proportional to the pressure gradient multiplied by the flow-stream cross-sectional area. The total volumetric flow rate is the sum of flow rates in all channels and equals the volumetric expansion rate of the aspiration bulb. Assuming that the expansion of the aspiration bulb is constant over a short period of time, if the available flow-stream cross-sectional area is halved by a permanent valve stopping fluid flow in one channel of a two channel system, then the pressure gradient and flow rate in the remaining channel will double.

In this circumstance, it may be useful to split the flow path just upstream of a valve, and use two valves instead of one, to reduce the flow rate, so that the response of the valve material will be sufficient to stop flow. This embodiment is illustrated in device 160 of FIG. 4, where one main flow path 161 is split into two paths 162 that encounter one valve 163 each, which may then recombine to connect to the aspiration bulb 164.

As described above, temporary valves can be used to aliquot the sample into multiple reaction chambers. FIG. 5 illustrates a multiple reaction chamber system or device 165 that also includes an ampule 178 encasing a secondary liquid in well 177 and a puncture lever 176 to actuate the delivery of the secondary liquid in well 177 to all of the reaction chambers 170.

To operate device 165 in FIG. 5, the user presses the integrated aspiration bulb 166, then places the tip 167 of the device 165 in the liquid source that is being tested. The user then releases pressure on the aspiration bulb 166. Similar to device 140 of FIG. 3, the liquid sample is aspirated though the flow-through or isolation valve 168 and into the reaction chambers 170, where flow is stopped by temporary valves 175. At this point the isolation valve 168 seals and no more liquid flows until puncture lever 176 is actuated. When the liquid enters reaction chambers 170 it also flows through and fills wells 169 just upstream of the reaction chambers 170. During the ‘incubation’ period between the time isolation valve 168 seals and the lever 176 is actuated, liquid in wells 169 is able to dissolve any reagents that may be present in the wells 169, in addition to the sample reacting with reagents that may be in the reaction chambers 170.

Once the puncture lever 176 of device 165 is actuated, the ampule 178 breaks, releasing the encased secondary liquid reagent in well 177, and a new air flow path is opened into the device 165, allowing liquid to advance in the system of device 165. As in device 140 of FIG. 3, a separate air channel and permanent valve can be used to bathe the ampule 178 in the sample liquid according to another embodiment. But, these features are not shown in FIG. 5 for clarity.

Once the ampule 178 is broken, the sample in wells 169 that have dissolved any reagent located there advances into the reaction chambers 170. The sample that has been in the reaction chambers 170 is washed out of the reaction chambers 170 through the first bank of temporary valves 175, and into the first bank of dead-volume washing wells 171.

There, advancement of fluid flow is again temporarily halted by the second bank of temporary valves 174. Eventually the second bank of temporary valves 174 also give-way and fluid flow resumes until all final dead-volume washing wells 172 have been filled and flow is permanently stopped by permanent valves 173. As fluid advances into wells 172, the secondary liquid reagent in well 177 from the ampule 178 has been able to wash through reaction chambers 170.

The circuit design of device 165 shown in FIG. 5 illustrates a multi-step assay including a liquid reagent delivery mechanism by which many complicated chemical or biological assays can be advantageously achieved. The only component of this processing circuit that may be particularly challenging to design is how long exactly the second set of temporary valves 174 should hold for. This temporary valve 174 timing parameter is, of course, application-specific and methods for controlling this time have been described above.

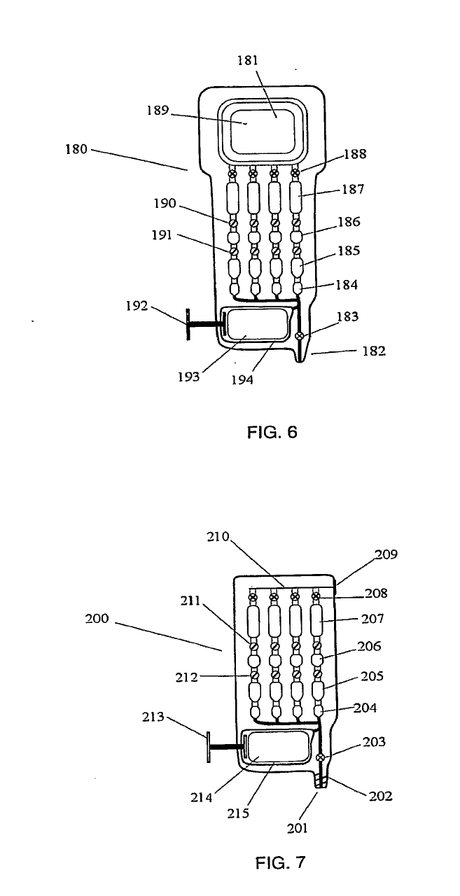

FIG. 6 illustrates another embodiment of a device 180 with similar functionality to the device 165 of FIG. 5 but where, instead of a breakable ampule 178, a bladder 194 encasing a secondary liquid in well 193 is used. Instead of a puncture lever 176, a plunger 192 is employed to burst the bladder 194 and dispense the integrated secondary liquid in well 193 into the reaction chambers 185. In this case the aspiration force drawing the secondary liquid from well 193 into the reaction chambers 185 may not be needed, and a pressure release hole 189 could be added to the bulb structure 181 as shown if desired. Instead of a bulb, a fitting for a syringe or pipette could be integrated into the device and the aspiration force could be generated by an external source (not shown) according to other embodiments of the present invention. Device 180 may further include integrated aspiration bulb 181, tip 182, permanent valves 188, dead-volume washing wells 186, 187, temporary valves 190, 191, wells 184, and flow-through or isolation valve 182 as shown in FIG. 6.

Alternatively, as is shown on device 200 in FIG. 7, an inlet fitting, or as shown a syringe connection 202, could be integrated into the inlet 201 for dispensing the sample into the device 200. In this case, there may not be an aspiration force available to draw in the secondary liquid 214, so a plunger design may be necessary. The inlet fitting 202 may not just be for a syringe, but for any type of sample collection and dispensing device. The only requirement is that enough pressure is available for dispensing the liquid into the device 200 properly, but not too much pressure as to blow through the temporary 211 and 212, isolation 203 or permanent 208 valves. The pressure may be from a syringe displacement, from bulb or pipette aspiration, from gravity or the pressure head from a column of liquid, from a hydraulic or pneumatic pump, or from an electrokinetic source according to various embodiments of the present invention. It is also possible that a filter (not shown) could be connected at the inlet 201, as described above, or a pressure regulator (not shown) or bleed valve (also not shown), if the main line pressure from which the sample is taken is expected to interfere with valve or device function.

A plunger, such as 213 on device 200 in FIG. 7, may be an integrated part of the device 200, or it could be a component of an external instrument (not shown). A plunger 213 could be actuated manually or automatically. It could just press on the outside of the device 200, such as on a flexible wall, or feed-through the outer wall of the device 200 and press on the ampule or bladder 215 in well 214, directly. As shown in FIG. 7, device 200 may further include wells 204, reaction chambers 205, dead-volume washing wells 206, 207, air duct 210 and exit port 209.

The timing of actuation of plunger 192 of device 180 in FIG. 6, or of plunger 213 of device 180 in FIG. 7, could also help simplify the design of the temporary valves in each device, such that they will not need to withstand pumping pressure for a specific pre-determined amount of time, since the pumping pressure can be controlled separately.

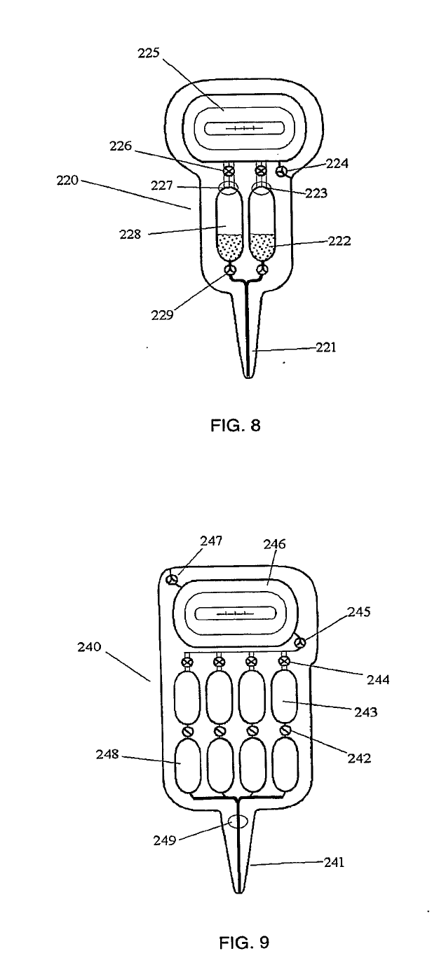

Liquid Reagents and Check-Valves: Another method of storing liquid in a reaction kit is to store the liquid directly in the reaction chambers and use check-valves to retain the liquid and prevent the liquid from leaking or dispensing out of the reaction chambers when an integrated aspiration bulb is pressed. This is illustrated in device 220 of FIG. 8. The check-valves 229 will be placed or designed to allow flow into the reaction chambers 228, but not out. To prevent liquid stored in the reaction chambers 228 from reacting with, or ‘spoiling’ the valves 226 that may be downstream of the reaction chambers, such as if the kit is inverted, various means can be employed around the location marked as 223 and 227. These include the use of narrow flow paths for viscous liquids, capillary breaks, temporary valves, or some other device or technique. A capillary break is a structure that, due to the presence of strong positive or negative capillary forces, prevents the further flow of liquid until and external perturbation (such as a pressure force stronger than the capillary force) is applied to the system. Check-valves with an appropriate ‘crack-pressure’ may also be used, which will prevent most instances of accidental flow or leakage, but still allow flow when significant aspiration or dispensing force is applied.

Check-valves may be formed into the kit during the manufacturing process, or inserted into the kit during a secondary assembly process. There are many kinds of check-valves, such as ball-valves, duck-bill valves, umbrella valves, or flapper valves. Many of these can be made in sizes to keep the total kit size small. Examples of useful check-valves for this type of application are manufactured by Minivalve International.

Device 220 shown in FIG. 8 is operated by the user holding device 220 in an upright position, such that the tip 221 or device inlet 221 is pointed down, and the integrated aspiration bulb 225 is up. The user then presses the bulb 225. The enclosed air, which might otherwise be dispensed out of the device 220 through the device inlet 221, is prevented from doing so by check-valves 229. Instead the air is dispensed out of device 220 through the check-valve 224 and its associated flow path to the outside of device 220. The tip 221 is then placed into the liquid to be tested (not shown). When the pressure on the bulb 225 is released, the test liquid is aspirated into reaction chambers 228, where it mixes and reacts with the liquid reagent 222 already present in the reaction chambers 228. Air does not leak into the bulb 225 through one-way check-valve 224. The bulb 225 can be continually actuated until the reaction chambers 228 are full. Any liquid leaking out of the reaction chambers 228 will be prevented from entering the bulb region due to the permanent valves 226.

Additional Uses of Check-valves: Device 240 in FIG. 9 illustrates the use of upstream check-valves 247 and downstream check-valves 245 of an integrated aspiration bulb 246. These check-valves 245, 247 allow the single stroke of the aspiration bulb 246 to be smaller than the total volume needed to fill the reaction chambers 248 of the device 240, or to drive liquid movement within the device 240 to complete its designed function. Upstream check-valve 247 allows air to be dispensed out of the device 240, but not into the device 240. Downstream check-valve 245 allows air to be aspirated into the bulb 246 when the bulb 246 is released, but does not allow air to be dispensed out of the bulb 246. Due to the compressibility of air and potential unwanted liquid movement caused by inaccurate device operation or accidental actuation of the liquid driving source (whether integrated bulb or external syringe or something else) check-valves 245, 247 may be placed in several strategic areas. Another such location where it may be desired to place a check-valve 245, 247, to allow one-way flow of liquid into the device 240, is at point 249 (shown circled).

Device 240 of FIG. 9 is operated by the user inserting tip 241 into the sample liquid and continually pumping the integrated aspiration bulb 246 until all reaction chambers 248 are filled. The reaction chambers 248 can be reliably filled due to the use of temporary valves 242. The bulb 246 can then be actuated again until all secondary reaction chambers 243, or dead-volume wash chambers 243 are filled. Liquid will not leak into the bulb 246 due to the presence of permanent valves 244. Due to air not being dispensed through the device 240 when the aspiration bulb 246 is pressed, by the function of one-way check-valves 245, 247, this device 240 could also hold liquid reagents in its wells 243, 248, if the liquid reagents could be reliably retained or contained within their wells 243, 248 such as by the use of capillary breaks.

Check-valves 245, 247, such as in the configuration as shown in device 240 of FIG. 9, may also be used to correlate bulb actuation with fluid advancement in a multi-step assay. Both the volume of the bulb 246 and fluid processing circuit could be designed to allow good correlation between number of bulb actuation cycles, such as whole integer numbers, and processing steps. For example, the user could be instructed to actuate the bulb 246 once to load the sample, wait a specified time for sample incubation, then actuate the bulb 246 two more times to wash and load a secondary reagent, wait, then actuate three more times to complete the assay. In this case the temporary and other valves may serve to both distribute and control liquid flow, but to also act as pressure barriers and as a compensation mechanism when bulb actuation does not exactly match fluid processing steps.

With the capability of correlating bulb actuation strokes with fluid advancement, the actual time response of temporary valves can be relaxed somewhat. Also, the timing and number of bulb actuation strokes would be a very straightforward function for a simple machine or piece of equipment to perform. This would eliminate the need for a piece of equipment to have an integrated pump, which would need to make a very good contact with a disposable cartridge to function properly. A simple machine could also actuate a lever to puncture a bladder or press a plunger to deliver a single or multiple on-board liquid reagents to a reaction area. The operation a user may need to perform to run a panel of sophisticated biological or chemical assays, including quantitative ELISAs, may be just as simple as squeezing and releasing a bulb to load a sample into a cassette, then placing the cassette into an inexpensive instrument that performs additional bulb actuations, puncture lever or plunger actuations if needed, incubation if needed, and optical or electronic measurements if needed.

The bulb design can be optimized to closely correlate with desired flow rates, pressure gradients and stroke volumes for optimal device function. Stroke volume can be controlled and variation between users minimized by instructing the user to push until their finger bottoms-out on the base of the bulb, or some artificial barrier within the bulb. As shown previously in EQ. 1, pressure and flow rates are, in part, a function of re-expansion of the aspiration bulb. While the expansion rate may be constant over a short period, it is definitely not constant over the total stroke cycle. The durometer, stiffness or hardness of the bulb material can be optimized for desired pressure gradients or suction force, and volumetric flow-rate profile during a stroke cycle. The material of the bulb can be optimized for the same effect. The use of ribs, thick and thin regions of the bulb can be optimized to vary the pressure gradient that the bulb, generates as it is re-inflating. Suitable bulb materials capable of this optimization include, but are not limited to, low-density polyethylene (LDPE), high-density polyethylene (HDPE), polypropylene (PP), urethane rubber, and others.

In many applications most reagents can be stored in a dry format directly within the device. However, in many high-sensitivity, optical, biological, enzymatic or other assays, unreacted components may need to be removed from the reaction, or measurement, area to prevent interference with the detection of a desired reaction product. This may best be done using an integrated secondary liquid that is stored outside the reaction chambers.

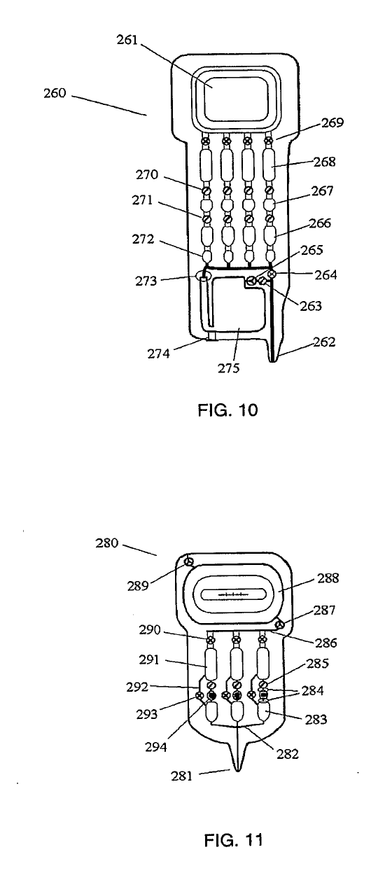

Device 260 of FIG. 10 illustrates a method for retaining a secondary liquid reagent within a reagent storage well, rather than within an ampule or bladder. For optimal device function, the device 260 should be held upright, such that the integrated bulb 261 is up and the tip 262 of the device 260 is pointed down. The user presses the integrated aspiration bulb 261 and places the tip 262 into the liquid sample being tested (not shown). The user then releases pressure on the bulb 261. The sample is aspirated into wells 272 and reaction chambers 266 through the isolation valve 264. The sample is not drawn into liquid reagent storage well 275 due to the temporary valve 263. When the bulb 261 is initially pressed, the liquid reagent stored in well 275 is not dispensed out due to the presence of the one-way check-valve 265, which only allows flow into well 275. The flow of liquid sample into the device 260 is distributed among the reaction chambers 266 and stopped from further advancement by temporary valves 271.

At this point isolation valve 264 seals and prevents further aspiration of liquid or air through device 260. After a period of time, temporary valves 271 release and all remaining aspiration force is directed through point 273 and used to aspirate the secondary liquid reagent in well 275 into the wells 272 and reaction chambers 266. Fluid advances and is distributed into wells 267 by the use of temporary valves 270. Eventually, temporary valves 270 release and liquid advances into dead-volume wells 268, where the liquid is prevented from leaking into the bulb 261 due to the permanent valves 269. The movement of liquid into the dead-volume wells 268 serves to wash-out any unbound reagents in reaction chambers 266 by the secondary liquid reagent from well 275.

In certain applications, it may be desirous to place an additional check-valve (with an appropriate crack-pressure), temporary valve, or capillary break at point 273 to assist in retaining the liquid stored in well 275 from leaking out, and to prevent the initial sample liquid from somehow leaking into well 275. To reduce the instances of trapping bubbles in device 260, it is desirous that the liquid stored in well 275 be as close to the point 273 and temporary valve 263 as possible, and that the initial sample liquid that is loaded is also brought very close to temporary valve 263 and point 273.

A secondary liquid reagent can be loaded into well 275 through access port 274, which is eventually sealed or capped off once the product manufacturing and assembly is completed. Methods of filling and sealing well 275 with a secondary liquid reagent will be within the skill of one of ordinary skill in the art.

It will be understood that check-valve 265 and temporary valve 263 may be somewhat redundant, and unnecessary for some applications. But, both valves 263 and 265 are shown in the illustrated embodiment, which will ensure proper device 260 functioning for other applications.

The methods for delivering an integrated secondary liquid reagent to reaction chambers involving an ampule, bladder, or liquid retained in a well have been described individually above. According to other embodiments of the present invention, multiple integrated liquid reagents may be integrated and delivered to reaction chambers 266 in series, using various combinations of the three methods described. In some cases an additional isolation valve 264, temporary valve 263, 270, 271 or check-valve 265 may be needed depending on the configuration of the device 260 and number of integrated liquids being delivered.

Storing and retaining a liquid within liquid storage wells of a sample processing system such as those disclosed herein offers another useful capability of priming valves or reaction chambers with a known liquid prior to filling the system with the unknown sample. For example, the response of temporary valves may vary depending on the composition of the sample fluid aspirated into a test kit. For example, if the kit is to be used for either salty or fresh water, the rate at which the temporary valve material dissolves into the water may vary considerably. If the precision and accuracy of the analysis cannot tolerate the variation in temporary valve response that may occur, it may be useful to prime the temporary valve with a liquid of known composition, where the response of the valve may be more reliable.

A test kit design with such a feature is illustrated by device 280 of FIG. 11. The three wells 294 (shown in gray) may be filled with a priming liquid used to prime the three temporary valves 285. To operate device 280 the user presses the integrated aspiration bulb 288 while placing the tip 281 of the device 280 into the fluid to be analyzed. As the bulb 288 is depressed, air is prevented from flowing through the common air duct 286 and into device 280 due to check-valve 287, and instead exits through check-valve 289. Once pressure on the bulb is released, the priming liquid stored in wells 294 is immediately aspirated into temporary valves 285, initially sealing temporary valves 285 and initiating the valve degradation process. However, fluid is still aspirated into the device 280 due to the parallel flow paths 292 connecting the dead-volume wash wells 291 to the reaction chambers 283, bypassing the sealed temporary valves 285 and priming liquid storage wells 294. Once the sample fluid fills the reaction chambers 283 through sampling channel 282, its flow is stopped due to the permanent valves 293. The mechanism of action and response of materials used for permanent valves 293 are typically less susceptible to response variations caused by variations in the sample liquid.

Once the temporary valves 285 dissolve and re-open, flow resumes into dead-volume wash wells 291, where flow is stopped by permanent valves 290.

The priming liquid may be retained in wells 294 by the use of additional temporary valves, check-valves, or capillary valves, if needed, at points 284 on the inlet and outlet of the wells 294.

This inventive method of retaining liquid and delivering liquid to a specific region prior to sample delivery, may also be useful to prime reaction chambers, such as to dissolve protective coatings on regions where biomolecules have been immobilized to ensure they respond properly once the sample is delivered according to other embodiments of the present invention.

Temporary Valves as Pressure Switches: The typical mechanism of a temporary valve is that of the valve material initially swelling and then dissolving in the fluid it encounters. At a minimum its response is that of dissolving in the system fluid. As shown in EQ. (1) earlier in this specification, once the flow of fluid is stopped in one channel, the flow rates and pressure gradients in remaining channels increases. An increase is pressure gradient may have the effect of driving fluid into the temporary valve material more forcefully than it may do otherwise, which may have the effect of causing the dissolution of valve material to happen more rapidly than at a lower pressure gradient. If there is no remaining aspiration pressure due, for example, to a pressure release hole in an aspiration bulb being uncovered, then a temporary valve may act effectively as a permanent valve. Alternatively, if the pressure increases, the temporary valve may release more quickly and, in effect, have some characteristics of a pressure switch.

This pressure switch effect may be enhanced by causing the valve material to be more finely ground, or in smaller granule sizes than usual, which increases the material surface area and makes it dissolve more rapidly. The pressure switch mechanism may be reduced by increasing valve material granule size, thus reducing the material surface area and reducing it solubility.

BioChemical Assays: An assay is a general term referring to a procedure where the concentration of a component part of a mixture is determined. Most of the reactions described in this disclosure can be referred to as assays. However, in the following text, an assay is more specifically referred to as a measurement procedure involving a biological component, or a biochemical reaction. The biological component could be the component that's presence is being determined, or it could be used to facilitate the measurement of a non-biological component. The biological component could be an antigen, antibody, protein, enzyme, nucleic acid or something else. An example of a biological component used to measure the presence of a non-biological component, would be the use of enzymes or antibodies to measure the presence of a heavy metal in solution. The assays could be standard immunological assays, such as ELISAs (enzyme-linked immuno sorbent assay) or a protein assay, such as BCA (Bicinchoninic acid assay), or something similar.

There are far too many biochemical assays to provide illustrations or device designs for each. Many assays categories follow similar procedural steps. However, even in this case the importance of each step, the robustness of the assay to step variations, the volumes needed for washing, the compositions and concentrations of particular reagents, developer enzymes, tracer antibodies, and the like, vary considerably and are application specific.

Numerous assays have been rendered to ‘test-strip’ designs, or lateral-flow chromatography, that use a single or multiple paper layers laden with specially formulated reagents. In the instances where important biochemical assays are available in this format, they are usually inexpensive to manufacture, accurate, easy to use, and provide a very beneficial function. However, it can take a considerable amount of time and a considerable amount of money spent in research and development before a fairly straightforward ‘liquid-based’ biochemical assay can be rendered to this platform. They are also usually just qualitative assays, and not quantifiable, due to the lack of control of fluid volumes that may be involved. The advantage of the technology described in this disclosure is that many of these important assays, and many others than cannot be rendered to the test-strip platform, can be commercialized far more quickly, and far less expensively, using a combination of dried and liquid-based reagents. The reagents are often in the same format and configuration in which they were originally developed, and panels of assays can be configured into the same device, with many checks and controls to improve reliability. They can also be designed for consumer-level use, and can be either semi-quantifiable, or highly quantifiable depending on the design and detection method used.

As mentioned, the assays described here use a combination of both liquid and dried reagents. Technology for dry storage of biochemical agents is well developed for many applications.

A complex ELISA protocol is shown here:

1. Sample Delivery

2. Incubate

3. Wash

4. Conjugate or tracer antibody delivery

5. Incubate

6. Wash

7. Substrate or developer enzyme delivery

8. Stop reagent delivery

This complex ELISA protocol assumes a starting point of wells or reaction chambers pre-loaded with adsorbed antibodies and treated to eliminate non-specific binding.

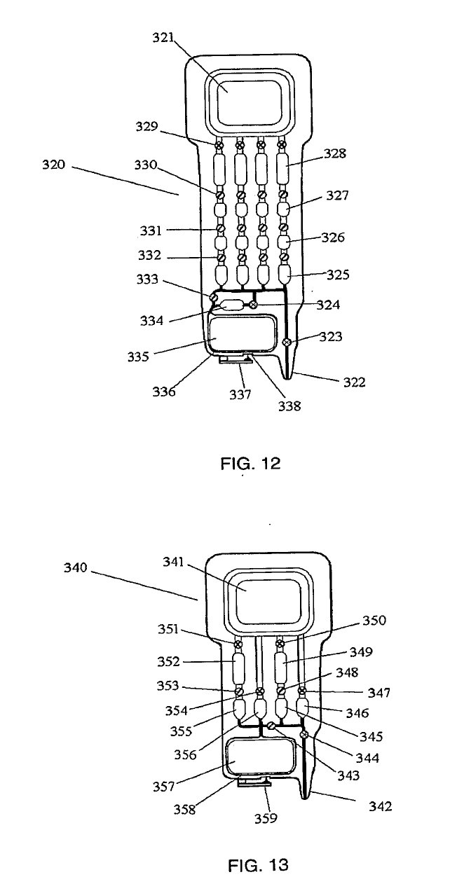

FIG. 12 illustrates a device 320 configured for performing this complex ELISA assay. The following process illustrates its operation, including both manual (MANUAL) and automatic (AUTO) steps:
1. User presses bulb 321, places tip 322 of device into a liquid sample (not shown), and then releases bulb 321. (MANUAL)
   a. Sample is loaded Into reaction chambers 325 where target antigens In the sample bind to immobilized antibodies (not shown) (AUTO).
   b. Temporary valves 332 seal, causing distribution of the sample into all reaction chambers 325 and preventing further fluid flow (AUTO).
   c. Flow-through valve 323 seals, stopping any further aspiration through the device inlet at tip 322 (AUTO).
   d. Sample is allowed to incubate in the reaction chambers 325 until the next manual step is performed (AUTO).
2. User presses puncture lever 337, piercing device wall 338 and breaking liquid-filled ampule 336 (MANUAL).
   a. First panel of temporary valves 332 release (AUTO).
   b. Remaining suction force on the bulb 321 aspirates secondary liquid in well 335 into system where it is directed by the temporary valve 333 into well 334, through the flow-through valve 324 and into the reaction chambers 325 (AUTO).
   c. Second panel of temporary valves 331 distribute the secondary liquid 335 into the reaction chambers 325 evenly, then seal to prevent further fluid flow downstream (AUTO).
   d. Secondary liquid 335 serves to wash out unbound sample from reaction chambers 325 (AUTO).
   e. Conjugate or tracer antibody (not shown) stored dry in well 334 is dissolved into secondary liquid in well 335 (AUTO).
   f. Temporary valves 331 release causing renewed flow of secondary liquid from well 335 into system, delivering conjugate or tracer antibodies into reaction chambers 325 (AUTO).
   g. Third panel of temporary valves 330 seal, causing even distribution of conjugate into reaction chambers 325 and preventing further liquid flow downstream (AUTO).
   h. Flow-through valve 324 seals (AUTO).
   i. Temporary valve 333 releases (AUTO).
   j. Bound sample and conjugate (not shown) are allowed to incubate in the reaction chambers 325 (AUTO).
   k. Temporary valves 330 release, drawing remaining secondary liquid 335 into reaction chambers 325, this time bypassing well 334, but flowing through the released temporary valve 333 (AUTO).
   l. Flowing secondary liquid 335 washes out any remaining unbound sample or conjugate from the reaction chambers 325 (AUTO).
   m. Substrate or developer enzyme (not shown), either a component of the secondary liquid 335, or alternatively stored dried in the reaction chamber 325 and finally released, or stimulated to release by a component of the secondary liquid 335, is delivered to or made available in the reaction chamber 325 (AUTO).
   n. Dead-volume wells 328 fill, permanent valves 329 seal, distributing remaining secondary liquid 335 into and through all reaction chambers 325 and preventing any further flow within the device (AUTO).
   o. Substrate or developer enzyme continues to generate signal until a stop reagent (not shown) is released from its encapsulated form within the reaction chamber 325 (AUTO).
3. User reads reaction results in reaction chambers 325 (MANUAL).

Device 320 allows a very complicated process with sophisticated valve function and reagent release timing. However, other embodiments of the present invention are contemplated with a few modifications that may improve its reliability. For example, after the user loads the sample the device could be placed within an instrument (not shown) that uses a plunger to press a bladder, rather than a piercing lever to break an ampule. The timed plunger pressure on the bladder, together with the function of the temporary valves, may improve fluid flow and sample incubation timing and device reliability. An instrument could also provide heat to bring the device to an elevated temperature for incubation and to possibly stimulate the release of stored reagents. The instrument could also use optical components to more accurately determine reaction results.

If the assay is very robust and includes reagents that are highly specific, a less sophisticated device can be used. Such a device 165 is shown in FIG. 5.

In device 165 there is no washing step between the sample incubation and conjugate or tracer antibody delivery. Also the sample, containing the analyte of interest (not shown), is used to release the conjugate in the conjugate wells 169. This may cause the analyte of interest in the sample to pre-bind to the conjugate, which, in some cases, may limit its ability to specifically bind to the immobilized antibody (not shown) in the reaction chamber 170. However, unconjugated sample is already in the reaction chamber 170 and incubating at the time the conjugate is released, and it is assumed that a surplus of conjugate is used so that plenty of conjugate is still available when it does finally reach the reaction chamber 170 in the next automatic fluid step.

The device 165 in FIG. 5 has the advantage that different conjugate or tracer antibodies can be stored in the four conjugate wells 169 and used in each reaction chamber 170, if desired, and there are fewer valves and fluid manipulation steps, which improves device reliability. There are also fewer places where bubbles can be trapped, which may interfere with device operation.

Another device 340, shown in FIG. 13, also uses a combination of downstream (relative to the reaction chamber 346) and upstream (relative to the reaction chamber 346) fluid control features, or valves, to provide sophisticated sample processing. However, in this design the valves are used to create different processing conditions, or liquid delivery steps, for each reaction chamber.

When the integrated aspiration bulb 341 is pressed, and the tip 342 of the device 340 is placed in the test liquid, and then the pressure on the integrated bulb 341 is released, sample is drawn into the device 340. The test liquid or sample flows through the isolation valve 344 and into reaction chambers 345 and 346. The temporary valve 343 prevents the sample from entering reaction chambers 355 and 356. Next, the isolation valve 344 seals. The operator then pushes the plunger 359 breaking the ampule 358, releasing the secondary liquid reagent within well 357. The secondary reagent in well 357 fills reaction chambers 355, 356 and then reaction chambers 345 and 346 once temporary valve 343 releases, but the permanent valves 354 and 347 prevent the secondary liquid from well 357 from washing the initial liquid from reaction chambers 356 and 346. Temporary valve 348 allows the sample to incubate in reaction chamber 345, then it releases and allows the sample to be washed out into the dead-volume well 349 by the secondary liquid 357. Temporary valve 353 allows the secondary liquid from well 357 to incubate in reaction chamber 355, and then be washed out into dead-volume well 352 by the secondary reagent 357. Liquid advancement in reaction chambers 355 and 345 and dead-volume wells 352 and 349 is stopped by permanent valves 351 and 350.

In this way four different conditions are generated in each of the four reaction chambers 355, 356, 345 and 346, which may be useful for a competitive ELISA, or other application. In this application it is assumed that reagents are stored in the reaction chambers for the specific reactions of interest.

Sample Concentration or Filtering: In many applications it may be useful to filter a large volume of liquid sample, and then to pass the filtrate into a reaction and detection system. This is useful for ‘trace-element’ detection, for analytes that may only be present in very low concentrations, when very poor or inaccurate measurements would be made on a ‘raw’ or unfiltered or un-concentrated sample.

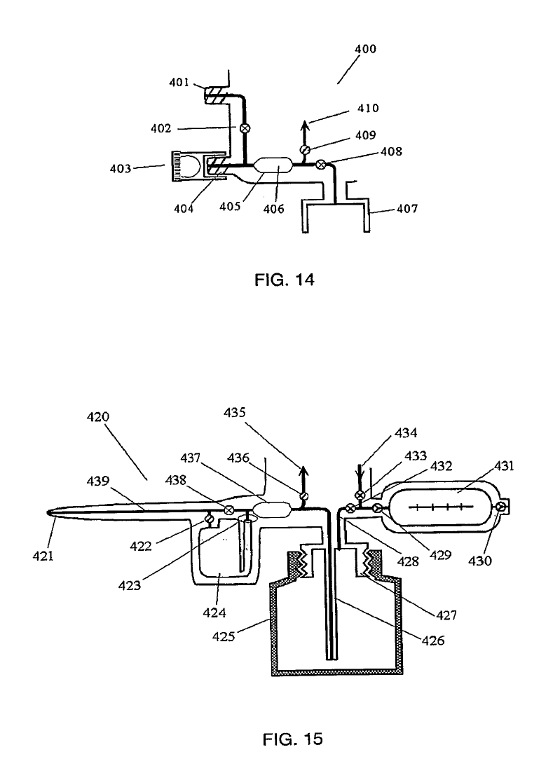

An example of an integrated fluid circuit using reactive or responsive valving to accomplish sample filtering or concentration is device 400 shown in FIG. 14. According to this design, a large volume of liquid is delivered to device 400 through inlet port 401. The liquid flows through isolation valve 402 and passes through a ‘concentration’ or filtering region 405 that contains a membrane filter, or various other capturing means 406, such as chromatography beads or resins designed to adsorb the analyte of interest, or beads or surfaces with immobilized antibodies or antigens, which are design to bind with the analyte of interest. This liquid is diverted from the main flow channel 410 leading to downstream reaction chambers (not shown) by temporary valve 409. The ‘dilute’ sample flows through isolation valve 408 and exits the device through outlet 407. The sample liquid also pushes air out through inlet 404, where it is prevented from leaking due to the use of a ‘bubble removal cap’ 403. After a sufficient amount of sample has passed through the concentration region 405, the isolation valve 408 closes, as does the isolation valve 402, and the temporary valve 409 releases. A secondary liquid (not shown) is delivered into the device by connecting a syringe or other dispensing source (not shown for clarity) onto inlet 404, after the bubble cap 403 has been removed. This secondary liquid is designed to elute or release the captured analyte, and deliver it to the main flow channel 410 into the downstream reaction chambers (not shown).

According to another embodiment of device 400 of FIG. 14, a second inlet, such as 404, may not be needed, nor is the isolation valve 402 always necessary, such as if there is only one inlet or if a cap is placed on inlet 401 after the dilute sample has been delivered. Also, dilute sample outlet 407 can be of many different configurations, such as a cap to be placed over an external waste collection receptacle (not shown), an integrated waste collection receptacle, an outlet with no fitting at all, or any other suitable outlet according to other embodiments of the present invention. Also, the concentration or filtering region 405 may have two outlets instead of one, according to another embodiment. One outlet may lead to isolation valve 408 and device outlet 407, and one outlet may lead to temporary valve 409 and the main flow channel 410.

Device 420 of FIG. 15 illustrates a test kit similar to device 400 of FIG. 14, except that the dilute sample delivery and filtration, and release agent delivery, are all achieved by the use of an integrated or externally connected aspiration bulb.

In device 420 the aspiration bulb 431 has check-valves 429 and 430 on the pump Inlet and outlet, respectively. These check-valves 429 and 430 prevent air from being pushed into the system by the pumping action of bulb 431. When the bulb 431 is actuated, sample liquid is drawn into device 420 through inlet 421. The fluid flows through the main flow channel 439, through the isolation valve 438, into the concentration region 437, through outlet 426 and into the waste collection receptacle 425. Flow is prevented from entering the well 424 containing the release reagent due to the temporary valve 422. Flow is prevented from entering a (generic) downstream reaction system (at 435) due to temporary valve 436.

As sample fluid fills the waste receptacle 425, which is either an integrated component of 420 or connected to 420 by the threaded connection point 427, the fluid will eventually reach channel 428 and re-enter the system. However, permanent valve 432 stops further flow along this flow path, and prevents fluid from entering aspiration bulb 431.

At this point all remaining aspiration force is directed onto flow path 434, drawing fluid into the reaction system 435 through the temporary valve 436. This temporary valve 436 may release as a matter of its timed degradation, or it could be designed to function as a pressure switch and open once flow is stopped through the flow path leading into the waste receptacle 425. Also at this point, isolation valve 438 should have sealed, redirecting flow through the temporary valve 422 and release agent in well 424. The release agent in well 424 is then drawn through the concentration region 437, eluting any bound analyte and delivering it to the downstream reaction system 435. Release agent in well 424 and any remaining sample being drawn into the inlet 421 continues to flow until it encounters permanent valve 433, and the process is complete.

Another temporary valve, check-valve, or capillary valve may be placed at location 423 to prevent premature release agent movement, according to other embodiments of the present invention.

What is claimed is:

1. A process for performing a chemical or biological reaction on a fluid sample, comprising:
providing a fluid analysis device, comprising:
at least one reaction chamber containing one or more reagents;
an inlet for receiving a fluid;
one or more fluid channels leading from the inlet to the reaction chambers;
valves connected to the at least one reaction chamber directly or through additional channels or chambers, the valves comprising a material configured to allow air to pass through the valve and configured to stop flow of the fluid through the valve upon contact with the fluid;
one or more exit channels connected to the valves to allow the flow of air out of the device until the valves are closed;
delivering fluid into the at least one reaction chamber of the fluid analysis device either by dispensing fluid into the fluid analysis device through the inlet, or by aspirating fluid into the fluid analysis device by generating a suction force at the one or more exit channels; and
allowing the aspiration or the dispensing to continue until all the valves leading to the one or more exit channels are closed, thereby ensuring all of the at least one reaction chambers are filled with the fluid, causing reagents stored within the reaction chamber to react with the fluid.

2. The process according to claim 1, further comprising modifying at least one component of the at least one reagent in at least one of the reaction chambers to increase reagent solubility in the fluid.

3. The process according to claim 2, wherein modifying includes at least one of:
adding reagents designed to provide an exothermal reaction;
adding reagents to cause an insoluble organic molecule to be converted into an organic salt;
adding components to cause the reagents to disintegrate thereby increasing reagent surface area;
adding rapidly soluble components to cause the reagents to become porous thus increasing reagent surface area;
freeze-drying or performing lyophilization of reagents to increase porosity; and
forming the reagents into thin sheets thereby increasing reagent surface area.

4. The process according to claim 1, wherein at least one component of the at least one reagent in the at least one reaction chamber has been modified to delay its solubility in the fluid.

5. The process according to claim 4, wherein modifying includes at least one of:
coating of the at least one reagent with a material that dissolves in the fluid over time or in response to a change in condition of the fluid,
encapsulating the reagent with a capsule that dissolves in the fluid over time or in response to a change in condition of the fluid,
embedding the reagent in a soluble or swellable matrix, the matrix swelling or dissolving in the fluid over time or in response to a change of condition of the fluid.

6. The process according to claim 5, wherein the change in condition is caused by the reaction of the at least one reagent present in the at least one reaction chamber or by the delivery of another fluid possessing reagents or characteristics that generate the change in condition.

7. The process according to claim 3, wherein modifying the at least one reagent in the at least one reaction chamber comprises a basic salt and p-dimethylaminobenzylidene rhodanine useful for reverse titration of cyanide in water.

8. The process according to claim 5, wherein modifying the at least one reagent in the at least one reaction chamber comprises potassium iodide embedded in a hydrogel matrix useful for titration of ethanol in solution.

9. An apparatus, comprising:
an inlet for receiving a fluid;
an isolation valve comprising a material configured to allow fluid to flow past it before it responds to or reacts with the fluid sufficiently enough to stop further fluid flow through it;
a fluid channel connecting the inlet to the isolation valve;
one or more reaction chambers, each comprising at least one entrance channel and one exit channel; and
a fluid channel connecting the isolation valve to the entrance channels of each reaction chamber.

10. The apparatus according to claim 9, further comprising a well including an enclosure for encasing the fluid and an inlet to the well being connected to the fluid channel at a point downstream of the isolation valve, but upstream of the entrance channels of each reaction chamber.

11. The apparatus according to claim 10, further comprising a lever disposed on the apparatus and configured to puncture or break the enclosure encasing the fluid, thus releasing the encased fluid.

12. The apparatus according to claim 10, further comprising a plunger configured to compress and rupture the enclosure encasing the fluid, thus releasing the encased fluid.

13. The apparatus according to claim 12, wherein the plunger is further configured to apply pressure to the enclosure encasing the fluid reagent, either manually or automatically, to dispense the fluid reagent out of the well inlet channel into the fluid channel connected upstream of the isolation valve.

14. The apparatus according to claim 10, further comprising an outlet channel connecting to the well containing the encased fluid, the outlet channel leads to a valve, the valve comprising a material configured to allow air to pass through the valve and configured to stop flow of the fluid through the valve upon contact with the fluid.

15. An apparatus, comprising:
an inlet for receiving a fluid;
a well comprising an entrance channel and at least one exit channel, the well further containing a material configured for capture or collection of an analyte of interest from the fluid entering the apparatus;
a fluid channel between the inlet and the entrance channel of the well;
an isolation valve connecting to an outlet of the well, the isolation valve comprising a material configured to allow the fluid to flow past it before it responds to or reacts with the fluid sufficiently enough to stop further fluid flow through it; and
a temporary valve connecting to an outlet of the well, such valve comprising a material configured to initially stop fluid flow through it, but eventually dissolve or break down such that fluid flow can resume after a specified period of time.

16. A process for filtering or concentrating a fluid sample through an analyte capture region, comprising:
providing a fluid analysis device, comprising:
an inlet for receiving a fluid;
a well comprising an entrance channel and at least one exit channel, the well further containing a material configured for capturing or collecting an analyte or analytes of interest from the fluid;
a fluid channel connecting the inlet to the entrance channel of the well;
an isolation valve connecting to an outlet of the well, the isolation valve comprising a material configured to allow the fluid to flow past the material before the material responds to or reacts with the fluid sufficiently enough to stop further fluid flow through the isolation valve; and
a temporary valve connecting to an outlet of the well, the temporary valve comprising a second material configured to initially stop fluid flow through the second material, but eventually dissolve or break down such that fluid flow can resume after a specified period of time;
delivering the fluid into the fluid analysis device through the inlet into the well; and
delivering a subsequent fluid into the well, wherein the subsequent fluid is configured to release or elute the analyte or analytes from the material.

17. A device configured to allow different fluid delivery conditions for reaction chambers contained within the device comprising at least one of the following:
one or more upstream sections disposed upstream of the reaction chambers that containing one or more materials that respond to the fluid to either temporarily or permanently impede further flow of the fluid or air through the one or more upstream sections; and
one or more downstream sections disposed downstream of the reaction chambers containing one or more other materials that respond to the fluid to either temporarily or permanently impede further flow of the fluid through the downstream sections.

18. An apparatus, comprising:
an inlet for receiving a fluid;
a fluid channel connecting the inlet to a junction at which junction the fluid channel splits into at least two branches;
at least one of the branches leading to an isolation valve comprising a material configured to allow the fluid to flow past the material before the material responds to or reacts with the fluid sufficiently enough to stop further fluid flow through the isolation valve;
at least one of the branches leading to a temporary valve comprising a second material configured to initially stop the fluid flow through the second material, but eventually dissolve or break down such that fluid flow can resume after a specified period of time;
a well containing a liquid, the well having a well inlet and a well outlet;
a channel connecting the well inlet to the temporary valve;
one or more reaction chambers, each containing at least one entrance channel and one exit channel;
a second fluid channel connecting the isolation valve to the entrance channels of the one or more reaction chambers; and
a third fluid channel connecting the well outlet to the entrance channels of the one or more reaction chambers.

19. The apparatus according to claim 18, further comprising a check-valve connected to the well inlet in series with the temporary valve, the check-valve is configured to allow liquid or air flow into the well inlet, but not out of the well inlet.

20. The apparatus according to claim 18, further comprising a valve connected to the well outlet located in between the well outlet and the third fluid channel, the valve selected from the group consisting of:
a check-valve designed to allow flow of liquid out of the well outlet, but not into the well outlet,
a valve comprising a material configured to initially stop fluid flow through it, but eventually dissolve or break down such that fluid flow can resume after a specified period of time, and
a valve designed to retain liquid within the well by the use of either positive or negative capillary forces.

21. The apparatus according to claim 20, wherein the check-valve further requires a specific pressure differential across it such that it will not open and allow fluid flow until specific conditions are generated by closure of the isolation valve.

22. The apparatus according to claim 18, further comprising a port leading from the outside of the apparatus into the well, the port designed to facilitate the loading of a liquid into the well, the port configured for sealing after the liquid is loaded into the well.

23. An apparatus, comprising:
an inlet for receiving a fluid;
one or more series of one or more wells, each series comprising at least one terminating well, the terminating well being the final downstream well in the one or more series of one or more wells, the terminating well comprising an entrance channel and an exit channel; and
a valve connected to the exit channel of each terminating well, the valve comprising a material configured to allow air to pass through the valve and configured to stop flow of the fluid through the valve upon contact with the fluid, each valve further possessing an air duct designed to allow air to pass through the valve.

24. The apparatus according to claim 23, further comprising an aspiration bulb with one or more inlets, the one or more inlets connected to the air ducts of each valve and configured to draw a vacuum at its one or more inlets until the valve or valves connected to it are closed.

25. The apparatus according to claim 24, further comprising an outlet connected to the aspiration bulb, the outlet vents to outside of the apparatus through a check-valve, the check-valve is configured to allow air to pass out of the aspiration bulb, but not into the aspiration bulb.

26. The apparatus according to claim 25, wherein the aspiration bulb is configured to have a single inlet and the apparatus further comprising a main air duct connecting the air ducts of each valve to the single inlet of the aspiration bulb, the main air duct connecting to the aspiration bulb inlet through a check-valve, which valve is configured to allow air to pass into the aspiration bulb inlet, but not out of the aspiration bulb inlet.

27. The apparatus according to claim 24, wherein at least one of the wells in the one or more of the series contains a liquid.

28. The apparatus according to claim 27, wherein the liquid containing well further comprises a check-valve on a well inlet of the well, the check-valve configured to allow air or liquid into the well inlet, but not out of the well inlet.

29. The apparatus according to claim 28, wherein the liquid containing well, if not a terminating well, further comprises a valve on a well outlet of the well, the valve being one of the following types:
a check-valve designed to allow flow of liquid or air out of the well outlet, but not into the well outlet,
a valve comprising a material configured to initially stop fluid flow through the material, but eventually dissolve or break down such that fluid flow can resume after a specified period of time, or
a valve designed to retain liquid within the well by the use of either positive or negative capillary forces.

30. The apparatus according to claim 25, wherein the aspiration bulb is configured to allow a total size of the apparatus to be smaller with the check-valve connected to the outlet of the aspiration bulb than would be possible without the check-valve connected to the aspiration bulb in order for the apparatus to operate correctly according to its desired function.

31. The apparatus according to claim 25, wherein a stroke volume of the aspiration bulb is configured to correlate with a step-wise movement of fluid within the apparatus to facilitate the function of a multi-step chemical or biological process.

32. The apparatus according to claim 24, wherein the aspiration bulb is configured with a selected material composition, material properties, and physical dimensions to correlate with various pressure gradients, stroke volumes and flow rates necessary to facilitate the movement of fluid within the apparatus to facilitate the function of a chemical or biological process.

33. The apparatus according to claim 23, further comprising one or more additional wells connected upstream of the terminating well entrance channel, the additional wells further comprising entrance and exit channels, and at least one of the additional wells further comprising a valve on its exit channel, the valve comprising a material configured to initially stop fluid flow through the material, but eventually dissolve or break down such that fluid flow can resume after a specified period of time.

34. The apparatus according to claim 33, wherein at least one of the one or more additional wells contains a liquid, the liquid either encased in an ampule or bladder, or retained in the additional well by a check-valve.

35. The apparatus according to claim 34, wherein such number of additional wells with valves connecting to the terminating wells are configured to facilitate liquid movement and reactions in a multi-step biological or chemical assay.

36. The apparatus according to claim 35, wherein such multi-step chemical or biological assays comprise washing out unbound reagents from the reaction chambers.

37. The apparatus according to claim 35, wherein such multi-step chemical or biological assays include an Enzyme-Linked Immuno Sorbent assay (ELISA).

38. The apparatus according to claim 35, wherein such multi-step chemical or biological assays comprises at least one of: a bicinchoninic acid assay (BCA), or a protein assay.

39. A method for temporarily or permanently stopping fluid flow within a device, comprising:
causing a liquid to enter a flow path through a device;
the flow path branching into at least two flow paths, each path connecting to its own valve, each valve comprising the same of one of the following two materials:
in the case where the valves are designed to permanently stop fluid flow, the valve material is configured to allow air to pass through the valve and configured to permanently stop flow of the fluid through the valve upon contact with the fluid;
in the case where the valves are designed to temporarily stop the flow of fluid the valve material is configured to initially stop fluid flow through it, but eventually dissolve or break down such that fluid flow can resume after a specified period;
the liquid reacting with each valve material located along the one or more flow paths, the liquid and the material generating a product that either temporarily or permanently impedes further flow of the liquid through the device, according to selected valve material.

40. A process for performing a multi-step chemical or biological assay on a fluid sample, comprising:
providing a fluid analysis device, comprising:
one or more reaction chambers containing one or more reagents;
an inlet for receiving a fluid;
one or more fluid channels leading from the inlet to the one or more reaction chambers;
valves connected to the reaction chambers directly or through additional channels or chambers, the valves comprising a material configured to allow air to pass through the valve and configured to stop flow of the fluid through the valve upon contact with the fluid;
one or more exit channels connected to each of the valves to allow the flow of air out of the device until the valves are closed;
an aspiration source connected to the exit channels of the valves configured to allow fluid to be aspirated out of the reaction chambers through the valves, but not into the reaction chambers through the valves, the aspiration source having selected dimensions and configuration to define a precise volume of liquid for each actuation stroke of the aspiration source to advance fluid through the apparatus to facilitate each step in the multi-step chemical or biological assay;
actuating the aspiration source either manually or automatically, or a combination of manual or automatic actuation, until all valves leading to the exit channels are closed.

41. A device configured to allow valves to be primed with a known fluid stored within the device comprising:
one or more sections containing a priming fluid of known composition;
one or more valves comprising a valve material configured to initially stop fluid flow through it, but eventually dissolve or break down such that fluid flow can resume after a specified period;
a fluid channel connecting the sections containing the priming fluid with the valves;
one or more bypass fluid channels connected to a point upstream of the one or more sections containing a priming fluid and connected downstream of the one or more valves;
the one or more bypass fluid channels connected through a valve comprising a valve material configured to allow air to pass through the valve and configured to stop flow of the fluid through the valve upon contact with the fluid.

42. An apparatus, comprising:
an inlet for receiving a fluid;
a fluid channel connecting the inlet to a junction at which junction the fluid channel splits into two branches;
the first branch leading to an isolation valve comprising a material configured to allow the fluid to flow past the material before the material responds to or reacts with the fluid sufficiently enough to stop further fluid flow through the isolation valve;
the second branch leading to a temporary valve comprising a second material configured to initially stop the fluid flow through the second material, but eventually dissolve or break down such that fluid flow can resume after a specified period of time;
a first well containing a liquid, the well having a well inlet and a well outlet;
a second well comprising an entrance channel and at least one exit channel, the well further containing a material configured for capture or collection of an analyte of interest from the fluid entering the apparatus;
the second branch further connecting the inlet of the first well to the temporary valve and the outlet of the first well to a point on the first branch upstream of the second well and downstream of the isolation valve;
a temporary valve connecting to an outlet of the second well, such valve comprising a material configured to initially stop fluid flow through it, but eventually dissolve or break down such that fluid flow can resume after a specified period of time.
an outlet of the second well leading to a waste collection region, the outlet of the waste collection region containing a valve comprising a material configured to allow air to pass through the valve and configured to stop flow of the fluid through the valve upon contact with the fluid.

43. A process for filtering or concentrating a fluid sample through an analyte capture region, comprising:
providing a fluid analysis device, comprising:
an inlet for receiving a fluid;
a fluid channel connecting the inlet to a junction at which junction the fluid channel splits into two branches;
the first branch leading to an isolation valve comprising a material configured to allow the fluid to flow past the material before the material responds to or reacts with the fluid sufficiently enough to stop further fluid flow through the isolation valve;
the second branch leading to a temporary valve comprising a second material configured to initially stop the fluid flow through the second material, but eventually dissolve or break down such that fluid flow can resume after a specified period of time;
a first well containing a liquid, the well having a well inlet and a well outlet;
a second well comprising an entrance channel and at least one exit channel, the well further containing a material configured for capture or collection of an analyte of interest from the fluid entering the apparatus;
the second branch further connecting the inlet of the first well to the temporary valve and the outlet of the first well to a point on the first branch upstream of the second well and downstream of the isolation valve;
a temporary valve connecting to an outlet of the second well, such valve comprising a material configured to initially stop fluid flow through it, but eventually dissolve or break down such that fluid flow can resume after a specified period of time.
an outlet of the second well leading to a waste collection region, the outlet of the waste collection region containing a valve comprising a material configured to allow air to pass through the valve and configured to stop flow of the fluid through the valve upon contact with the fluid.
delivering the fluid into the fluid analysis device by placing the inlet into the fluid and actuating an aspiration source drawing the fluid into the device until flow stops and the process is complete.
